(19) United States
(12) Patent Application Publication
(10) Pub. No.: US 2007/0270832 A1
(43) Pub. Date: Nov. 22, 2007

(54) Locking device and method, for use in a bone stabilization system, employing a set screw member and deformable saddle member

(75) Inventor: Jeffrey D. Moore, Olive Branch, MS (US)

Correspondence Address: HESLIN ROTHENBERG FARLEY & MESITI P.C., 5 COLUMBIA CIRCLE, ALBANY, NY, 12203, US

(73) Assignee: SDGI Holdings, Inc., Wilmington, DE (US)

(21) Appl. No.: 11/414,879

(22) Filed: May 1, 2006

(51) Int. Cl. A61F 2/30

(52) U.S. Cl. 606/61

(57) ABSTRACT

A set screw with a deformable saddle for use in securing a stabilization member within a bone stabilization system. The bone stabilization system includes a bone anchor, a stabilization member, a coupling mechanism and a locking device. The locking device is structured to engage the coupling mechanism and the coupling mechanism is configured to couple the stabilization member to the bone anchor. The locking device which is configured to thread into the coupling mechanism, includes a set screw connected to a saddle member. The saddle member is fabricated from a deformable material. The locking device is configured to frictionally secure the stabilization member within the coupling mechanism by the application of a compressive force.

### CROSS-REFERENCE TO RELATED APPLICATIONS/PATENTS

This application contains subject matter which is related to the subject matter of the following applications/patents, which are hereby incorporated herein by reference in their entirety:
“Multi-Axial Bone Attachment Assembly”, Coates et al., U.S. Ser. No. 10/870,011, filed Jun. 17, 2004, and published on Dec. 22, 2005 as Patent Application Publication No. US 2005/0283157 A1;
“Coupling Assemblies for Spinal Implants.”, Justis et al., U.S. Ser. No. 11/197,799, filed Jan. 31, 2006;

“Force Limiting Coupling Assemblies for Spinal Implants”, Justis et al., U.S. Ser. No. 11/112,221, filed Jan. 31, 2006; and
“Bone Anchor System Utilizing A Molded Coupling Member For Coupling A Bone Anchor To A Stabilization Member And Method Therefor”, Dewey, et al, U.S. Ser. No. ______, co-filed herewith (Attorney Docket No.: P23147.00).

### TECHNICAL FIELD

The present invention relates generally to orthopaedic implants used for the correction of spinal injuries or deformities, and more specifically, but not exclusively, concerns apparatuses for fixing a particular segment or level of the spine, to allow for deformity correction or healing thereof.

### BACKGROUND OF THE INVENTION

In the field of spinal surgery, it is known to place implants into vertebrae for a number of reasons, including: (a) correcting an abnormal curvature of the spine; (b) to maintain appropriate vertebral spacing and provide support for broken or otherwise injured vertebrae; and (c) to perform other treatments in the spinal column.

Typical spinal implant or bone stabilization systems utilize a rod as the support and stabilizing element. In such a system, a series of two or more bone fasteners are inserted into two or more vertebrae to be instrumented. A rod or other stabilizing device is then placed within or attached to the heads of the bone fasteners, or is placed within a coupling device that links the rod and the head of the bone fastener. The connections between these multiple components are then secured, thereby fixing a supporting construct to multiple levels in the spinal column.

To advance the state of orthopaedic implants, enhancement to such bone stabilization systems are believed desirable, and addressed herein.

### SUMMARY OF THE INVENTION

Connecting the stabilization member, the bone fastener and the coupling device together in a bone stabilization system can result in high forces being exerted onto the stabilization member, thereby increasing the potential for post-operative failure to occur. Thus, a need exists for the use of a deformable material for securing the stabilization member in a bone stabilization system. The introduction of a deformable material at the stabilization member and coupling device locking interface will reduce the stresses realized in the stabilization member, decrease the creation of surface stress risers on the stabilization member and enhance the level of securement within the bone stabilization system.

Currently, many locking mechanisms utilized in bone stabilization systems potentially induce component failure during in vivo post-operative loading because of the increase in the level of stresses placed on components as a result of certain implant designs and particular construct materials. Currently, the use of carbon fiber composite polymers and other materials that possess similar flexural moduli in the manufacturing of stabilization members is under development. An advantage that is realized when the stabilization member and components of the locking device are fabricated from similar or the same materials is the decrease or elimination of regions of stress concentrations. The invention described herein addresses the associated problems of the use of dissimilar construct materials by utilizing a deformable material in the fabrication of the locking mechanism. The use of like materials in fabricating the locking device and the stabilization member reduces the generation of stress risers, and thereby causes a decrease in the stress levels realized within the secured stabilization member.

Thus, the shortcomings of the prior art are overcome and additional advantages are provided through the provision of a locking device for use in a bone stabilization system, the bone stabilization system includes a bone anchor, a coupling mechanism and a stabilization member, wherein the coupling mechanism is configured to couple the stabilization member to the bone anchor, the locking device includes a set screw member and a saddle member. The set screw member may be configured to operatively engage with the coupling mechanism to secure the stabilization member within the coupling mechanism. The saddle member is fabricated from a deformable material and is attached to the set screw member. The saddle member includes a distal interface surface and a proximal interface surface, with the distal interface surface being shaped to partially surround the stabilization member when the stabilization member is placed within the coupling mechanism. The proximal interface surface of the saddle member is configured to couple to the set screw member.

The present invention provides, in another aspect, a bone stabilization system which includes a bone anchor, a stabilization member, a coupling mechanism and a locking device. The coupling mechanism is configured to operatively connect the bone anchor and the stabilization member. The locking device includes a set screw member configured to thread into the coupling mechanism and a saddle member. The saddle member is attached to the set screw member and is fabricated from a deformable material.

An enhanced aspect of the locking device for use in a bone stabilization system employing a set screw member and a deformable saddle member is the saddle member to be fabricated from the same material as the stabilization member. The material used to make the saddle member and the stabilization member is a type of plastic. The plastics used for fabricating the saddle member and the stabilization member are deformable and possess a flexural modulus that are either the same or substantially similar to each other. The plastics preferably used for manufacturing the saddle member and the stabilization member is a polyetheretherketone. A potential drawback to the use of polyetheretherketone is the material's susceptibility to breakage due to its inherent notch sensitivity.

The present invention provides, in another aspect, a method for stabilizing a spinal column by the use of a bone stabilization system. The bone stabilization system includes a bone anchor, a stabilization member, a coupling mechanism and a locking device with the coupling mechanism configured to couple the stabilization member to the bone anchor and the locking device being operatively associated to the coupling mechanism. The locking device includes a set screw member configured to engage the coupling mechanism and a saddle member coupled to the set screw member, wherein the saddle member is made from a deformable material. The method further includes positioning the stabilization member within the coupling mechanism and engaging the locking device to the coupling mechanism by threading the set screw member into the coupling mechanism. The set screw member is advanced into the coupling mechanism causing the saddle member to partially compress against the stabilization member and frictionally hold the stabilization member within the coupling mechanism.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 is a perspective view of one embodiment of a bone stabilization system, in accordance with an aspect of the present invention;

FIG. 2 is a perspective view of one embodiment of a coupling mechanism, in accordance with an aspect of the present invention;

FIG. 3 is a cross-section elevational view of the coupling mechanism of FIG. 2 taken along line 3-3, in accordance with an aspect of the present invention;

FIG. 4 is a perspective view of one embodiment of a locking device threadably engaged within the coupling mechanism of FIGS. 2 & 3, in accordance with an aspect of the present invention;

FIG. 5 is a side elevational view of the locking device and the coupling mechanism embodiment of FIG. 4, in accordance with an aspect of the present invention;

FIG. 6 is a side elevational view of the locking device embodiment of FIGS. 4 & 5, in accordance with an aspect of the present invention;

FIG. 7 is a distal perspective view of the locking device embodiment of FIGS. 4-6, in accordance with an aspect of the present invention;

FIG. 8 is a distal perspective view of one embodiment of a set screw member, in accordance with an aspect of the present invention;

FIG. 9 is a distal perspective view of one embodiment of a saddle member, in accordance with an aspect of the present invention;

FIG. 10 is a side elevational view of the saddle member of FIG. 9, in accordance with an aspect of the present invention; and

FIG. 11 is a side elevational view of one embodiment of a bone anchor, in accordance with an aspect of the present invention; and

FIG. 12 is a cross-section elevational view of the locking device of FIG. 6 taken along line 12-12, in accordance with an aspect of the present invention.

### BEST MODE FOR CARRYING OUT THE INVENTION

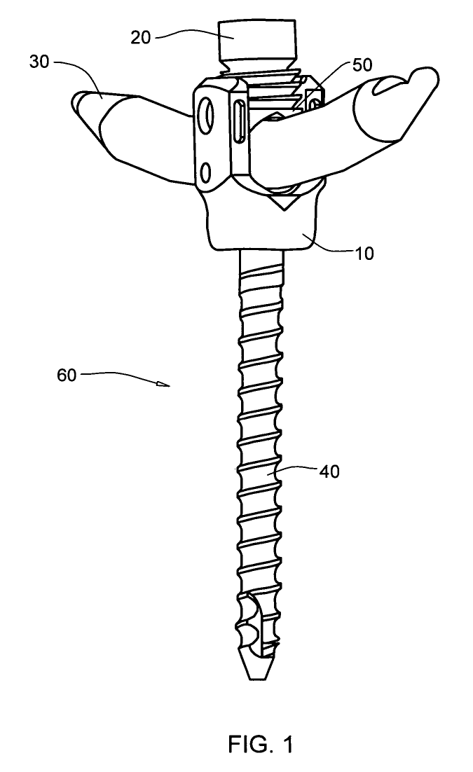

As depicted in FIG. 1, the general arrangement of a bone stabilization system 60, in accordance with an aspect of the present invention, includes a coupling mechanism 10, a locking device, comprising a set screw member 20 and a saddle member 50, a stabilization member 30, and a bone anchor 40. When used in the spine to secure multiple levels of the spinal column, a bone anchor 40 is placed within an individual vertebrae and a coupling mechanism 10 is then attached to each implanted bone fastener 40. Following placement of multiple bone anchors and coupling mechanisms, an appropriately dimensioned stabilization member 30, which spans the levels of the affected vertebral region, is placed within the coupling mechanisms 10 and secured in place employing multiple locking devices. As noted, the locking device includes set screw member 20 and saddle member 50. When the locking device is in use stabilization member 30 is frictionally held in place between coupling mechanism 10 and the locking device by saddle member 50. A locking device, with deformable saddle member 50, as described herein, reduces the resultant stresses realized in stabilization member 50 by decreasing the generation of surface stress risers when fixed within coupling mechanism 10 by being fabricated from the same or similar material as stabilization member 50 and having a concave distal interface surface that further deforms to the shape of stabilization member 50.

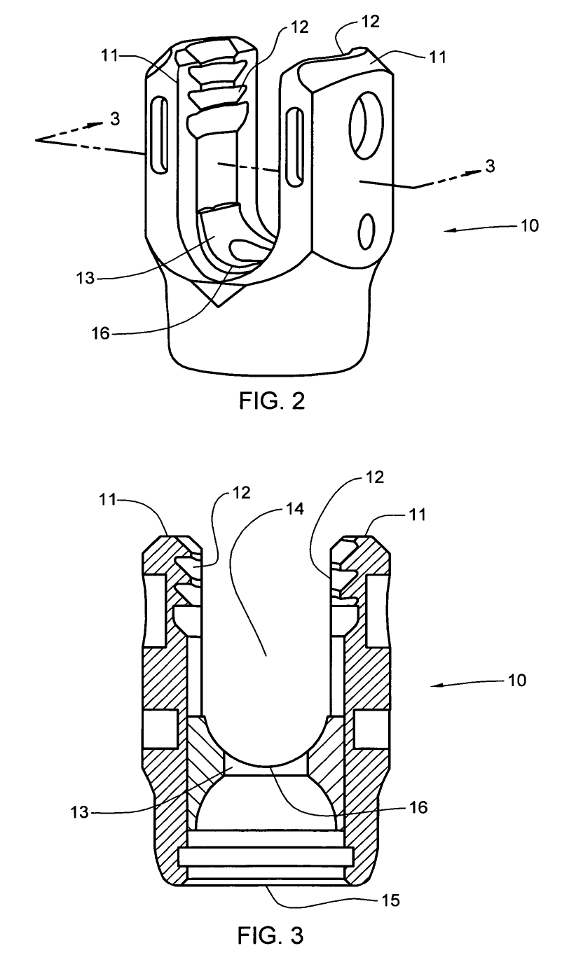

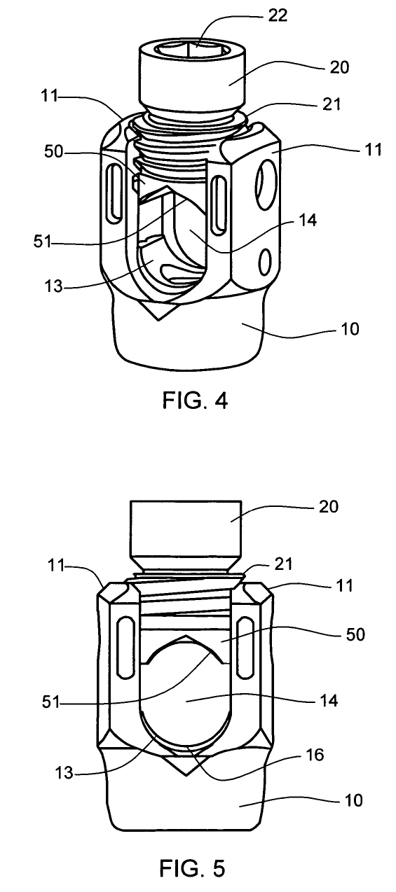

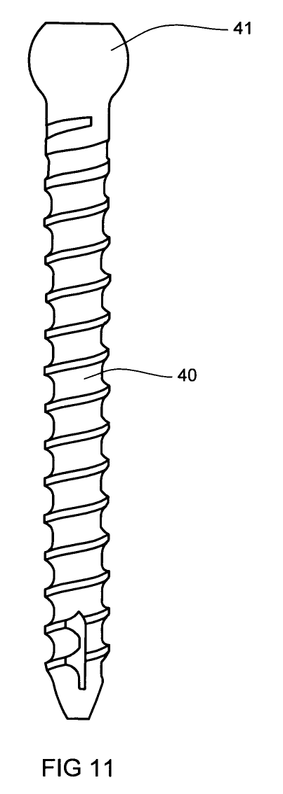

With reference to FIGS. 2 and 3, bone stabilization system 60 includes coupling mechanism 10 with a channel 14 defined by a seat 13 and a pair of coupling arms 11. Coupling arms 11 are disposed parallel and project in an upward manner from seat 13. Coupling arms 11, together with seat 13 form U-shaped channel 14 which is appropriately sized to receive stabilization member 30. The internal walls of coupling arms 11 include internal threads 12 or alternatively an internal cam surface (not shown) to engage external threads 21 of set screw member 20 (as shown in FIGS. 4-8). Typically, at least one through hole 15 is located directly below seat 13 in coupling mechanism 10. In one approach, bone anchor 40 (see FIG. 1) is inserted into hole 15 prior to the placement of stabilization member 30. The longitudinal axis of bone anchor 40 may be at a fixed angle relative to coupling mechanism 10 following insertion into hole 15 or be allowed to pivot within hole 15. Hole 15 may be counter bored, counter sunk, slotted, have a spherical seat, keyed or any combination or derivation of these manufacturing techniques, to allow the top portion of the anchor head 41 (see FIG. 11) to sit below the seat floor 16.

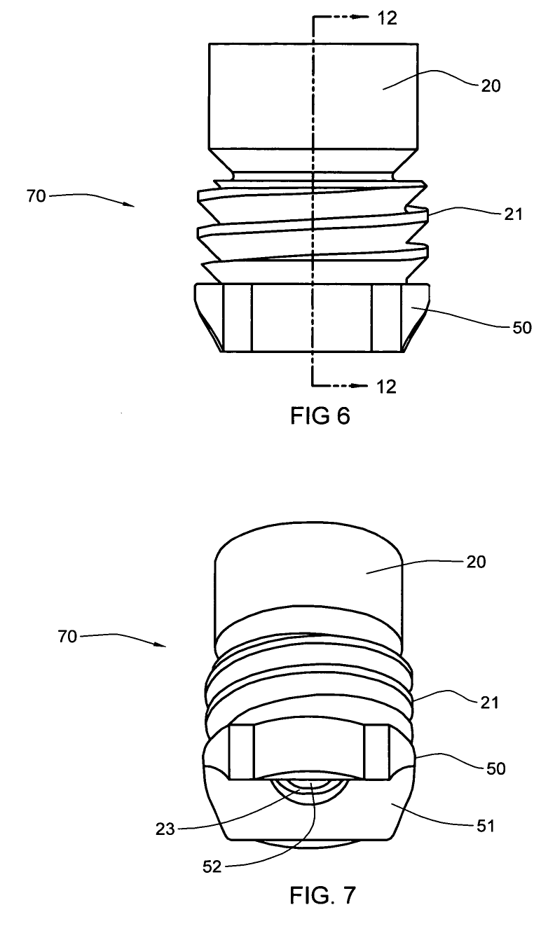

As shown in FIGS. 4 and 5, coupling mechanism 10 is illustrated as including at least one set screw member 20 threadably engaged with internal threads 12, although it is understood to those skilled in the art that other configurations are contemplated, including a set screw configured to include an external cam surface (not shown) that engages with an internal cam surface (not shown) located on the internal surface of coupling arms 11. In an unlocked position, the locking device 70 (see FIGS. 6 & 7) initiates engagement with internal threads 12 and allows stabilization member 30 to move freely within channel 14. When in a locked position, preferably the locking device 70 (see FIGS. 6 & 7) is substantially engaged with internal threads 12, resulting in a pressing engagement or a compressive force being applied across the distal interface surface 51 of saddle member 50 onto stabilization member 30.

Stabilization member 30 is typically shaped as an elongate and continuous orthopaedic implant, preferably in the shape of a rod. Alternative stabilization members may include, but are not limited to plates, bars, tethers, cables, elastic structures and dynamic stabilization members (not shown). Stabilization member 30 may be fabricated from a plastic material, preferably polyetheretherketone (PEEK) polymer. Alternatively, stabilization member 30 may be fabricated from a material selected from the group consisting of carbon fiber composite polymers, bio-compatible metals, shape memory metals, resorbable polymers, bio-inert polymeric materials, thermoplastic polymers, thermoset polymers and any combinations of these materials.

Referring to FIGS. 6 and 7, locking device 70 is shown and preferably includes set screw member 20 with external threads 21 for engagement with coupling member 10, and saddle member 50. As depicted in FIG. 6, saddle member 50 is connected to set screw member 20, adjacent to the distal end 25 of set screw member 20. As seen in FIG. 4, a tool recess 22 is located in the proximal end of set screw member 20 to facilitate the insertion of a tightening tool or instrument (not shown). FIG. 8 depicts set screw member 20 prior to being coupled to saddle member 50. A cylindrical locking nub 23 extends from distal end 25. Locking nub 23 is sized to allow for insertion into a hole located preferably in the central aspect of saddle member 50. Set screw member 20 may be fabricated from a titanium alloy, preferably the alloy Ti-6Al-4V. Alternatively, set screw member 20 may be fabricated from a material selected from the group consisting of CP titanium, cobalt-chromium, a 300 series stainless steel, carbon fiber materials, carbon fiber composites, resorbable polymers, bio-inert polymeric materials, thermoplastic polymers, thermoset polymers and any combination of these materials.

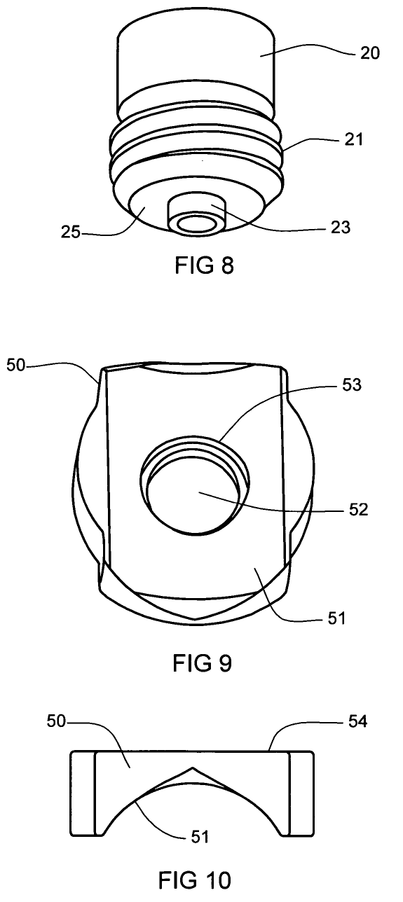

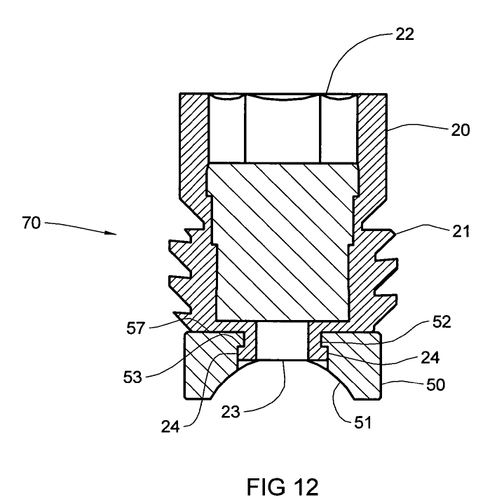

Saddle member 50 preferably includes a proximal interface surface 54 (see FIG. 10) that may contact distal end 25 following the securement of stabilization member 30 by the locking device 70 (see FIGS. 6 & 7) within coupling mechanism 10. As seen in FIGS. 9 and 10, saddle member 50 may include a distal interface surface 51 that is preferably configure to partially surround stabilization member 50. The shape and material of distal interface surface 51 allows saddle member 50 to elastically deform and frictionally secure stabilization member 30 when locking device 70 is threadingly advanced into coupling member 10. Typically, as shown in FIG. 9, the outer perimeter of distal interface surface 51 is rectangular shaped. As depicted in FIG. 10, the concave receiving channel is preferably located in the central aspect of distal interface surface 51, running generally parallel to the long axis of the rectangular outer perimeter. Saddle member 50 may include at least one hole 52 which preferably passes from the proximal interface surface 54 through to distal interface surface 51. Hole 52 may be located in the center area of saddle member 50. The centerline of hole 52 may be about normal to proximal interface surface 54. The inner diameter of hole 52 is preferably configured and dimensioned to receive locking nub 23. As shown in FIG. 9, hole 52 preferably includes a counter bore 53, wherein the depth of counter bore 53 relative to distal interface surface 51 is variable depending on the construct material and may range between 0.5 mm to 1.0 mm. As depicted in FIG. 12, counter bore 53 is configured and dimensioned to provide for the end of the flare 24 to remain below distal interface surface 51 and not contact the external surface of stabilization member 30 when locking device 70 is fully engaged and applying a load that frictionally secures stabilization member 30 within coupling member 10. Saddle member 50 is fabricated from a deformable plastic material, preferably the polyetheretherketone (PEEK) polymer. Alternatively, saddle member 50 may be fabricated from another deformable material selected from the group consisting of carbon fiber composite polymers, UHMWPE, shape memory metals, resorbable polymers, bio-inert polymeric materials, thermoplastic polymers, thermoset polymers and any combinations of these materials. Preferably, the material used to comprise saddle member 50 will have a flexural modulus that is equivalent or similar to the flexural modulus of stabilization member 30. The preferred range of the flexural modulus of saddle member 50 is from about 30 to 115 MPa.

As shown in FIG. 11, bone anchor 40 is typically configured as a bone screw, although, alternative bone anchors may be utilized including, but not limited to bone fixation posts (not shown), bone staples (not shown), hooks (not shown), and moveable head screws (not shown). It is understood to those skilled in the art that the bone anchor-coupling mechanism structure described is for example only and that other configurations may be used, including coupling mechanism 10 configured to be integrally coupled to bone anchor 40.

The assembly process for connecting saddle member 50 to set screw member 20 typically includes the steps of placing set screw member 20 on a flat surface with distal end 25 being upright. Proximal interface surface 54 is placed onto distal end 25. Saddle member 50 must be aligned to allow for locking nub 23 to pass through hole 52 and protrude above the edge of counter bore 53. Preferably, the next step in the assembly process is for a deforming load to be applied to the end of locking nub 23, thereby generating flare 24 on the end of locking nub 23 as shown in FIG. 12. Flare 24 contacts the internal shoulder of counter bore 53 as seen in FIG. 12 and substantially prohibits any axial translational movement of saddle member 50 relative to set screw member 20. Flare 24 does allow rotational movement of saddle member 50 relative to set screw member 20.

The steps of the method for stabilizing a spinal column includes, first providing bone stabilization system 60 consisting of bone anchor 40, stabilization member 30, coupling member 10 and locking device 70. Coupling device 10 may be constructed to couple stabilization member 30 to bone anchor 40. The locking device 70 is configured to operatively associate with coupling mechanism 10. The locking device 70 includes saddle member 50 connected to set screw member 20, with saddle member 50 being fabricated from a deformable material. The next step of the method is to preferably position stabilization member 30 into channel 14 located within coupling mechanism 10. The next step is to initiate engagement of locking device 70 to coupling mechanism 10. The last step of the method of stabilizing a spinal column would be to threadingly advance set screw member 20 downward into coupling arms 11 and cause saddle member 50 to contact and partially compress stabilization member 30 within channel 14. Preferably, locking device 70 is sufficiently advanced to frictionally hold stabilization member 30 within coupling mechanism 10. Although the preferred embodiments have been depicted and described in detail herein, it will be apparent to those skilled in the relevant art that various modifications, additions and substitutions can be made without departing from its essence and therefore these are to be considered to be within the scope of the following claims.

What is claimed is:

1. A locking device for use in a bone stabilization system, the bone stabilization system including a bone anchor, a coupling mechanism and a stabilization member, wherein the coupling mechanism is configured to couple the stabilization member to the bone anchor, the locking device comprising:
a set screw member operatively associated with the coupling mechanism for securing the stabilization member within the coupling mechanism; and
a saddle member attached to the set screw member, the saddle member being fabricated from a deformable material and including a distal interface surface and a proximal interface surface, wherein the distal interface surface is contoured to partially surround the stabilization member and the proximal interface surface is configured to couple to the set screw member.

2. The locking device of claim 1, wherein the deformable material comprises a first plastic material, and wherein the stabilization member comprises a second plastic material.

3. The locking device of claim 2, wherein the first plastic material comprises a first flexural modulus and the second plastic material comprises a second flexural modulus substantially the same as the first flexural modulus.

4. The locking device of claim 3, wherein at least one of the first flexural modulus and the second flexural modulus have a value ranging from 30 MPa to 115 MPa.

5. The locking device of claim 2 wherein at least one of the first plastic material and second plastic material comprises a polyetheretherketone material.

6. The locking device of claim 1, wherein the deformable material comprises at least one of a polymer, carbon fiber material, carbon fiber composite, thermoplastic polymer and thermoset polymer.

7. The locking device of claim 1, wherein the stabilization member is an elongate orthopaedic implant having a first end, a second end and a longitudinal axis extending therebetween, wherein when the locking device is in use the stabilization member is received within the coupling mechanism and the locking device engages the stabilization member along the longitudinal axis thereof.

8. The locking device of claim 1, wherein the stabilization member is fabricated from at least one of a notch-sensitive material and a breakable material.

9. The locking device of claim 1, wherein the set screw member comprises a biocompatible metal.

10. The locking device of claim 9, wherein the set screw member comprises a titanium alloy.

11. The locking device of claim 1, wherein the distal interface surface of the saddle member comprises a concave surface.

12. The locking device of claim 1, wherein the saddle member is configured with at least one hole extending between the proximal interface surface and the distal interface surface.

13. The locking device of claim 1, wherein the set screw member comprises a distal end, a proximal end, and a central axis extending between the distal end and the proximal end, wherein a locking nub extends from the distal end, and the proximal end is configured to facilitate coupling thereto by an insertion tool.

14. The locking device of claim 13, wherein the locking nub comprises a flare radially extending therefrom.

15. The locking device of claim 12, wherein the hole is counterbored relative to the distal interface surface of the saddle member to provide an internal shoulder sized to contact the radially extending flare from the locking nub and thereby limit movement of the set screw member relative to the saddle member.

16. The locking device of claim 15, wherein the saddle member and the set screw member are rotatably coupled to each other.

17. The locking device of claim 1, wherein the set screw member is configured to threadably engage the coupling mechanism, wherein threading of the set screw member into the coupling mechanism results in applying a load to the saddle member and securing the stabilization member between the saddle member and the coupling mechanism coupled to the bone anchor.

18. A bone stabilization system, the bone stabilization system comprising:
a bone anchor;
a stabilization member;
a coupling mechanism, wherein the coupling mechanism is configured to operatively connect the bone anchor and the stabilization member; and
a locking device, wherein the locking device operatively connects to the coupling mechanism to secure the stabilization member within the coupling mechanism, wherein the locking device comprises:
a set screw member configured to threadably engage the coupling mechanism; and
a saddle member attached to the set screw member, the saddle member being fabricated from a deformable material.

19. The bone stabilization system of claim 18, wherein the saddle member includes a distal interface surface and a proximal interface surface, wherein the distal interface surface is contoured to partially surround the stabilization member when the stabilization member is secured between the coupling mechanism and the locking device, and the proximal interface surface is configured to couple to the set screw member.

20. The bone stabilization system of claim 18, wherein the saddle member comprises a first deformable material, and wherein the stabilization member comprises a second deformable material.

21. The bone stabilization system of claim 20, wherein the first deformable material comprises a first flexural modulus and the second deformable material comprises a second flexural modulus substantially the same as the first flexural modulus.

22. The bone stabilization system of claim 21, wherein at least one of the first flexural modulus and the second flexural modulus have a value ranging from 30 MPa to 115 MPa.

23. The bone stabilization system of claim 20 wherein at least one of the first deformable material and second deformable material comprises a polyetheretherketone material.

24. The bone stabilization system of claim 18, wherein the deformable material comprises at least one of a polymer, carbon fiber material, carbon fiber composite, thermoplastic polymer and thermoset polymers.

25. The bone stabilization system of claim 18, wherein the stabilization member comprises an orthopaedic rod having a first end, a second end and a longitudinal axis extending therebetween, and wherein when the locking device is in use, the stabilization member is received within the coupling mechanism and the locking device engages the stabilization member along the longitudinal axis.

26. The bone stabilization system of claim 25, wherein the stabilization member is fabricated from at least one of a notch-sensitive material and a breakable material.

27. The bone stabilization system of claim 19, wherein the distal interface surface of the saddle member comprises a longitudinally extending concave channel.

28. The bone stabilization system of claim 19, wherein the saddle member is configured with at least one central hole extending between the proximal interface surface and the distal interface surface.

29. The bone stabilization system of claim 18, wherein the set screw member comprises a distal end, a proximal end, and a central axis extending between the distal end and the proximal end, wherein a locking nub extends from the distal end, and the proximal end is configured to facilitate coupling thereto by an insertion tool.

30. The bone stabilization system of claim 29, wherein the locking nub comprises a flare radially extending therefrom.

31. The bone stabilization system of claim 28, wherein the hole is counterbored relative to the distal interface surface of the saddle member to provide an internal shoulder sized to contact the radially extending flare of the locking nub and thereby limit movement of the set screw member relative to the saddle member.

32. The bone stabilization system of claim 31, wherein the saddle member and the set screw member are rotatably coupled to each other.

33. The bone stabilization system of claim 18, wherein the set screw member is configured to threadably engage the coupling mechanism, wherein threading of the set screw member into the coupling mechanism results in applying a load to the saddle member and securing the stabilization member between the saddle member and the coupling mechanism coupled to the bone anchor.

34. A method for stabilizing a spinal column, the method comprising:
providing a bone stabilization system comprising a bone anchor, a stabilization member, a coupling mechanism, and a locking device, wherein the coupling mechanism is configured to couple the stabilization member to the bone anchor, the locking device being operatively associated with the coupling mechanism, wherein the locking device further comprises a set screw member configured to threadably engage the coupling mechanism and a saddle member connected to the set screw member, the saddle member being fabricated from a deformable material;
positioning the stabilization member in the coupling mechanism;
engaging the locking device to the coupling mechanism; and
threadingly advancing the set screw member into the coupling mechanism, thereby causing the saddle member to contact the stabilization member and partially compress against the stabilization member in order to frictionally hold the stabilization member within the coupling mechanism.
